(19) United States
(12) Patent Application Publication
(10) Pub. No.: US 2025/0359816 A1
(43) Pub. Date: Nov. 27, 2025

(54) VIRTUAL REMOTE TELE-PHYSICAL EXAMINATION SYSTEMS

(71) Applicants: The United States Government as represented by the Department of Veterans Affairs, Washington, DC (US); BOARD OF REGENTS, THE UNIVERSITY OF TEXAS SYSTEM, Austin, TX (US)

(72) Inventors: Thiru Annaswamy, Hershey, PA (US); Balakrishnan Prabhakaran, Richardson, TX (US); Kevin Desai, San Antonio, TX (US); Ninad Arun Khargonkar, Richardson, TX (US)

(21) Appl. No.: 18/874,393

(22) PCT Filed: Jun. 13, 2023
(86) PCT No.: PCT/US2023/025158

§ 371 Date: Dec. 12, 2024

Related U.S. Application Data
(60) Provisional application No. 63/351,671, filed on Jun. 13, 2022.

(51) Int. Cl. A61B 5/00; A63F 13/56; G02B 27/01; G06V 40/20

(52) U.S. Cl. CPC A61B 5/4528; A61B 5/744; A63F 13/56; G02B 27/017; G06V 40/20

(57) ABSTRACT

Methods, systems, and apparatuses are described for estimating the force acting on at least one joint of a user while the user engages at least one virtual object within a virtual environment. Motion data associated with joint data of at least one joint of a user may be received from a sensor. The joint data may be used to determine force information. The force information may be used to determine user strength associated with the at least one joint.

### CROSS REFERENCE TO RELATED PATENT APPLICATION

This Application claims priority to U.S. Provisional Application No. 63/351,671, filed Jun. 13, 2022, which is herein incorporated by reference in its entirety.

### BACKGROUND

Assessing the strength in joints is a vital step of rehabilitation monitoring and general musculoskeletal examinations. In an in-person assessment, the strength of a joint is assessed using an isometric test which requires physical interaction between the physician and the patient. However, a considerable proportion of patients needing care do not live close by to a medical center, especially in remote non-urban areas. In addition, even with such infrastructure, close proximities may be inhibited by external factors, such as during a pandemic. Such challenges emphasize the need for tele-medicine and remote assessment procedures to ensure continued and accessible care. There exist several approaches for remote strength assessment with sophisticated methods in particular, such as motion capture, on-body sensors, and haptic devices lying on one end of the spectrum and audio-visual feedback via video-chat application on the other end.

The sensor-based approach involves placing on-body sensors on various joints, tracking parameters of interest such as velocity, accelerations, and muscle activations. However, such an approach requires additional technical experts in dealing with the sensors and their accurate placement. In addition, there is a tendency to impede natural motion and cause tangible discomfort to users. Forgoing on-body sensors, common motion capture methods require multiple specialized cameras which are calibrated across multiple viewpoints and hence cannot be readily set up in a patient's home for remote assessment. Haptics-based methods also suffer from similar challenges of expensive setup and coordinating several devices working in tandem. In addition, most of these systems focus on a live, synchronous version of assessment via video chat interaction and are not conducive to asynchronous use cases. However, there is an implicit need to go beyond the current standard for providing additional feedback to the physician. Widespread availability of cheap RGB+Depth (RGB-D) cameras offer an avenue of providing non-invasive tracking and enhanced levels of feedback without the hassle of calibration. Such cameras utilize the depth stream to provide the inference and tracking of human joints in the absence of any markers for the body segments.

With the use of RGB-D cameras, virtual reality systems and applications have incorporated 3D human models, such as personalized humanoid avatars representative of the user as captured by the RGB-D cameras. Virtual reality (VR) refers to a computer-generated environment which enables users to experience their physical senses and perception. VR has countless applications in myriad industries ranging from entertainment and gaming to engineering and medical science. For example, virtual environments can be the setting for interacting with a personalized avatar or a simulated surgery. VR experiences are rendered so as to be perceived by physical sensing modalities such as visual, auditory, haptic, somatosensory, and/or olfactory senses. In a similar vein, augmented reality (AR) refers to a hybrid environment which incorporates elements of the real, physical world as well as elements of a virtual world. Like VR. AR has countless applications across many different industries. The complementary nature of AR makes it well-suited to applications such as gaming, engineering, medical sciences, tourism, recreation, and the like. The use of 3D human models give users a better sense of immersion as they see details such as the dress the human is wearing, their facial emotions, etc. VR games, utilizing these 3D human models may be used to enhance the user experience and help in the remote assessment of a patient. Furthermore, the use of consumer depth cameras allow for a more natural interaction experience with an exercise game (exergame) system.

### SUMMARY

It is understood that both the following general description and the following detailed description are exemplary and explanatory only and are not restrictive.

Methods, systems, and apparatuses are described for rendering a personalized humanoid avatar within a virtual environment to assist in the strength assessment of a user's joints. The virtual reality scene may comprise the personalized humanoid avatar within the virtual environment. A VR device may comprise one or more sensors which may determine one or more of a position, orientation, location, and/or motion of the user within the virtual environment.

In an embodiment, are methods comprising performing, based on receiving calibration data from a sensor. a real-time camera-to-skeletal pose calibration, wherein the sensor comprises a RGB-D camera, causing a display to output a user interface within a virtual environment, wherein the user interface includes a gaming session control menu, wherein the gaming session control menu comprises an option to output a game to engage the user to interact with at least one virtual object to engage the user to move at least one specified portion of the user's body, receiving, from the sensor, motion data, wherein the motion data comprises motion data of user movements engaging the at least one virtual object during the game, wherein the motion data includes joint data, causing the display, based on the real-time camera-to-skeletal pose calibration, to output a personalized humanoid avatar of the user within the virtual environment, causing, based on applying the motion data to the personalized humanoid avatar, the personalized humanoid avatar to perform at least one motion, tracking. based on the joint data, an angle of at least one joint over a time duration from start to end of the user's movements while the user engages the at least one virtual object, determining, based on the tracked angle, a force estimation model estimating a force acting on at least one joint while the user engages the at least one virtual object during the game, determining, based on the force estimation model, an inference of user strength associated with the at least one joint, and sending, to a server, the force estimation model, wherein the server stores the force estimation model in a database associated with the user.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of the present description serve to explain the principles of the methods and systems described herein. To easily identify the discussion of any particular element or act, the most significant digit or digits in a reference number may refer to the figure number in which that element is first introduced.

FIG. 1 shows an example system;

FIG. 2 shows an example system;

FIG. 3 shows example movements;

FIG. 4 shows an example depth camera representation of a 3D skeleton of a user;

FIG. 5A-5B show an example scenes;

FIG. 6 shows an example scene;

FIG. 7 shows an example scene;

FIG. 8 shows an example process;

FIG. 9 shows a flowchart of an example process; and

FIG. 10 shows an example headset device.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including. but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the present methods and systems may take the form of a computer program product on a computer-readable storage medium (e.g., non-transitory) having processor-executable instructions (e.g., computer software) embodied in the storage medium. More particularly, the present methods and systems may take the form of web-implemented computer software. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These processor-executable instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These processor-executable instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Hereinafter, various embodiments of the present disclosure will be described with reference to the accompanying drawings. As used herein, the terms “user,” or “subject.” may indicate a person who uses an electronic device or a device (e.g., an artificial intelligence electronic device) that uses an electronic device.

Methods and systems are described for generating a personalized humanoid avatar of a user within a virtual environment to assist in the synchronous and asynchronous remote strength assessment of the user's joints in an interactive augmented reality setting. A VR device may comprise, or be in communication with, a camera, which may be, for example, any imaging device such as a RGB-D camera, a digital camera, and/or digital video camera. Throughout the specification, reference may be made to VR or a VR device. It is to be understand that VR and AR may be used interchangeably and refer to the same circumstances or devices. The camera may be associated with a field of view (e.g., a frame) representing an extent of the observable world that the camera may image. The VR device may utilize the camera to capture one or more image data while a user performs one or more movements in the field of view, process the image data, and generate motion data of the user's movements, including joint data associated with the user's joints as the user performs the different movements. The VR device may comprise, or be in communication with a display. For example, the display device may comprise a head-mounted device (HMD), a smartphone, a smart mirror, a monitor, a laptop, a tablet, a television, and the like. The display may output a user interface that may include a gaming session control menu. The gaming session control menu may include an option to output a game to engage the user to interact with at least one virtual object to engage the user to move a portion of the user's body. The VR device may receive motion data generated by the camera while the user interacts with the at least one object during the game. The display may output a personalized humanoid avatar of the user within the virtual environment. The VR device may cause the personalized humanoid avatar to perform at least one motion based on the motion data. The VR device may track an angle of the at least one joint while the user interacts with the at least one virtual object. The VR device may determine a force estimation model estimating a force acting on the at least one joint based on the tracked angle of the user's joint. Using the force estimation model, the VR device may determine an inference of the subject's strength associated with the at least one joint.

Lastly, the VR device may send the joint strength data to a server. The joint strength data may be associated with each user in order to improve the quality of the user's experience during the entire process and track the joint strength estimations for each user. For example, a user's physician may use the joint strength estimations of the user to make any further assessments regarding the user's joints.

In an example, the camera may require a calibration in order to compensate for a rendering perspective of the subject interacting with the VR device. The calibration of the camera may include a real-time camera-to-skeletal pose and a real-time floor calibration to estimate a floor plane of an environment detected in the calibration data.

The VR device may further include one or more cameras configured to capture images of the subject to generate the real-time 3D personalized humanoid avatar of the subject.

The VR system may comprise two mixed reality scenes that function like exercise games in providing an interactive and engaging element to the remote assessment procedure. The primary focus may be on the upper body joints due to ease of tracking and lower possibility of noise and occlusions (unlike lower body joints such as ankles). The VR system may target three upper-body joints of the elbow, wrist, and shoulder for a total of four movements. The camera may be used for motion tracking and 3D skeleton inference. The VR system may record the user motion for a specified joint in terms of the range of motion via the camera skeleton data and the time required to complete the motion via tracking hand gestures. The VR system may estimate a force value for the motion using an inverse dynamics solver which may then be transmitted to virtual objects inside the game environment for providing an in-game feedback mechanism to a physician performing the remote assessment.

Depending on the VR application, one or more virtual objects of varying size. shape, orientation, color, and the like may be determined. For example, in a VR game application, a virtual object may be determined. Spatial data associated with the one or more virtual objects may be determined. The spatial data associated with the one or more virtual objects may comprise data associated with the position in 3D space (e.g., x, y, z coordinates). For a given virtual object of the one or more virtual objects, the position in 3D space may comprise a position defined by a center of mass of the virtual object and/or a position defined by one or more boundaries (e.g., outline or edge) of the virtual object. The spatial data associated with the one or more virtual objects may be registered to spatial data associated with the center of frame. Registering may refer to determining the position of a given virtual object of the one or more virtual objects relative to the position of the center of frame. Registering may also refer to the position of the virtual object relative to both the position of the center of the frame and the position of the personalized avatar in the mixed reality scene. Registering the virtual object to the position of the center of the frame and/or the positions of any of the one or more physical objects in the virtual reality scene results in ensuring that a display of the virtual object in the virtual reality scene is made at an appropriate scale and does not overlap (e.g., “clip”) with any of the one or more virtual objects, or the avatar, in the virtual reality scene. For example, the spatial data of the virtual object may be registered to the spatial data of the center of the frame and to the spatial data of a table (e.g., one of the one or more physical objects). Such registration enables the virtual object to be displayed in the virtual reality scene so that the virtual object appears to rest on the table and does not overlap (e.g., “clip”) the table.

Movement of the VR device may cause a change in the virtual reality scene. For example, the VR device may pan, tilt, or rotate to a direction of the VR device. Such movement may impact the virtual reality scene and the personalized avatar and any virtual objects rendered therein. For example, if the VR device is tilted downward, the perspective within the virtual environment may rotate downward, akin to a person shifting his/her head downward. Likewise, if the VR device is tilted upward, the perspective within the virtual environment may rotate upward, akin to a person shifting his/her head upward. In an example, if the VR device is rotated leftward or rightward, the perspective within the virtual environment may rotate leftward or rightward, akin to a person rotating his/her head leftward or rightward.

Each of the constitutional elements described in the present document may consist of one or more components, and names thereof may vary depending on a type of an electronic device. The electronic device according to various exemplary embodiments may include at least one of the constitutional elements described in the present document. Some of the constitutional elements may be omitted, or additional other constitutional elements may be further included. Further, some of the constitutional elements of the electronic device according to various exemplary embodiments may be combined and constructed as one entity, so as to equally perform functions of corresponding constitutional elements before combination.

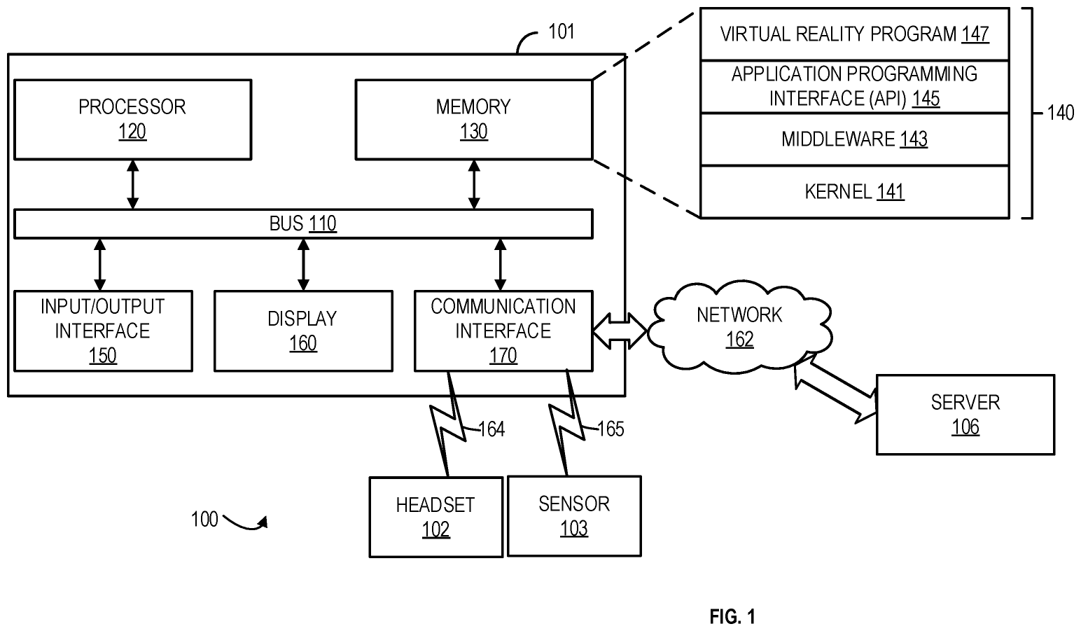

FIG. 1 shows an example system 100 including an electronic device (e.g., smartphone or laptop) configured for controlling one or more guidance systems of one or more other electronic devices (e.g., a headset device or sensor device) according to various embodiments. The system 100 may include an electronic device 101, a headset 102, one or more sensors 103, and one or more servers 106. The electronic device 101 may include a bus 110, a processor 120, a memory 130, an input/output interface 150, a display 160, and a communication interface 170. In an example, the electronic device 101 may omit at least one of the aforementioned constitutional elements or may additionally include other constitutional elements. The electronic device 101 may comprise, for example, a mobile phone, a smart phone, a tablet computer, a laptop, a desktop computer, a smartwatch, and the like.

The bus 110 may include a circuit for connecting the processor 120, the memory 130, the input/output interface 150, the display 160, and the communication interface 170 to 170 to each other and for delivering communication (e.g., a control message and/or data) between the processor 120, the memory 130, the input/output interface 150, the display 160, and the communication interface 170.

The processor 120 may include one or more of a Central Processing Unit (CPU), an Application Processor (AP), and a Communication Processor (CP). The processor 120 may control, for example, at least one of the processor 120, the memory 130, the input/output interface 150, the display 160, and the communication interface 170 of the electronic device 101 and/or may execute an arithmetic operation or data processing for communication. The processing (or controlling) operation of the processor 120 according to various embodiments is described in detail with reference to the following drawings. For example, the processor 120 may be configured to cause the headset device 102 to output a virtual reality game to the user, such as the virtual reality program 147 stored in the memory 130.

The memory 130 may include a volatile and/or non-volatile memory. The memory 130 may store, for example, a command or data related to at least one different constitutional element of the electronic device 101. In an example, the memory 130 may store a software and/or a program 140. The program 140 may include, for example, a kernel 141, a middleware 143, an Application Programming Interface (API) 145, and/or a virtual reality program (e.g., an “application”) 147, or the like, configured for controlling one or more functions of the electronic device 101 and/or an external device (e.g., the headset 102 and/or the one or more sensors 103). At least one part of the kernel 141, middleware 143, or API 145 may be referred to as an Operating System (OS). The memory 130 may include a computer-readable recording medium having a program recorded therein to perform the method according to various embodiments by the processor 120.

The kernel 141 may control or manage. for example. system resources (e.g., the bus 110. the processor 120, the memory 130, etc.) used to execute an operation or function implemented in other programs (e.g., the middleware 143, the API 145, or the virtual reality program 147). Further, the kernel 141 may provide an interface capable of controlling or managing the system resources by accessing individual constitutional elements of the electronic device 101 in the middleware 143, the API 145, or the virtual reality program 147.

The middleware 143 may perform, for example, a mediation role so that the API 145 or the virtual reality program 147 can communicate with the kernel 141 to exchange data. In addition, the middleware 143 may handle one or more task requests received from the virtual reality program 147 according to a priority. For example, the middleware 143 may assign a priority of using the system resources (e.g., the bus 110, the processor 120, or the memory 130) of the electronic device 101 to at least one of the virtual reality programs 147. For example, the middleware 143 may process the one or more task requests according to the priority assigned to the at least one of the application programs, and thus may perform scheduling or load balancing on the one or more task requests.

The API 145 may include at least one interface or function (e.g., instruction), for example, for file control, window control, video processing, or character control, as an interface capable of controlling a function provided by the virtual reality program 147 in the kernel 141 or the middleware 143.

The virtual reality program 147 may comprise two mixed reality scenes that function like exercise games in providing an interactive and engaging element to the remote assessment procedure. The primary focus may be on the upper body joints due to ease of tracking and lower possibility of noise and occlusions (unlike lower body joints such as ankles). The VR system may target three upper-body joints of the elbow, wrist, and shoulder for a total of four movements. A sensor device 103 may be used for motion tracking and 3D skeleton inference. For example, the electronic device 101 may receive motion data from the sensor device 103, while a subject is interacting with the virtual reality program 147. The virtual reality program 147 may record the user motion for a specified joint, based on the data (e.g., sensor skeleton data) received from the sensor device 103. in terms of the range of motion and the time required to complete the motion via tracking hand gestures. The virtual reality program 147 may estimate a force value for the motion using an inverse dynamics solver which may then be transmitted to virtual objects inside the game environment for providing an in-game feedback mechanism to a physician performing the remote assessment.

The input/output interface 150 may play a role of an interface for delivering an instruction or data input from a user or a different external device(s) to the different constitutional elements of the electronic device 101. Further. the input/output interface 150 may output an instruction or data received from the different constitutional element(s) of the electronic device 101 to the different external device.

The display 160 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. The display 160 may display, for example, a variety of contents (e.g., text, image, video, icon, symbol, etc.) to the user. The display 160 may include a touch screen. For example, the display 160 may receive a touch, gesture, proximity, or hovering input by using a stylus pen or a part of a user's body.

The communication interface 170 may establish, for example, communication between the electronic device 101 and an external device (e.g., a headset 102, a sensor device 103, or a server 106). For example, the communication interface 170 may communicate with the external device (e.g., the server 106) by being connected to a network 162 through wireless communication or wired communication. In an example, as a cellular communication protocol, the wireless communication may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), and the like. Further, the wireless communication may include, for example, a near-distance communication 164, 165. The near-distance communications 164, 165 may include, for example, at least one of Wireless Fidelity (WiFi), Bluetooth, Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. The wired communication may include, for example, at least one of Universal Serial Bus (USB), High Definition Multimedia Interface (HDMI), Recommended Standard-232 (RS-232), power-line communication, Plain Old Telephone Service (POTS), and the like. The network 162 may include, for example, at least one of a telecommunications network, a computer network (e.g., LAN or WAN), the internet, and a telephone network.

The headset 102 may comprise a head-mounted display (HMD) device that may include an optical element that may selectably turn on or off a view of an outside environment in front of a person's eyes. The headset 102 may be configured to execute the virtual reality program 147. Using the various sensors and modules, the headset may perform a real-time camera-to-skeletal pose and a real-time floor calibration to estimate a floor plane of an environment detected in the initial data. In an example, the subject may adjust his/her position to interact with virtual objects in the virtual environment in order to calibrate the sensors and/or the headset 102. Based on the calibration, the headset 102 may output a personalized humanoid avatar of the subject. In an example, the headset 102 may comprise a display device such as a television, a monitor, a laptop, or a tablet.

As an example, movement of the headset 102 may cause a change in the virtual reality scene. For example. the headset 102 may pan, tilt. or rotate to a direction of the headset 102. Such movement may impact the virtual reality scene and the personalized avatar and any virtual objects rendered therein. For example, if the headset 102 is tilted downward, the perspective within the virtual environment may rotate downward, akin to a person shifting his/her head downward. Likewise, if the headset 102 is tilted upward, the perspective within the virtual environment may rotate upward, akin to a person shifting his/her head upward. In an example, if the headset 102 is rotated leftward or rightward, the perspective within the virtual environment may rotate leftward or rightward, akin to a person rotating his/her head leftward or rightward.

In an example, the headset 102 may use the various sensor devices and modules to detect objects or obstacles in front of the subject as the subject is performing the game via the headset 102. The headset 102 may be configured to alert the subject of any potential objects that may pose a safety risk to the subject while the subject is performing the movements while playing the game using the headset 102. For example. objects such as pets or people could pose a safety issue as they come within the distance of the subject's movements. The headset 102 may be configured to use an object detection module for detecting objects (e.g., pets, people, etc.) within a radius of the headset 102. When the object (e.g., pet, person, etc.) moves into a field of view of a sensor of the headset 102, an alarm may be trigger to alert the subject of the object. For example, the headset 102 may output a pop-up panel within the image stream and a detection result, In an example, a predicted bounding box may be output identifying the object relative to the subject in the image stream output by the headset 102 to the subject.

The sensor device 103 may comprise one or more imaging, or image, capture devices such as one or more RGB-D cameras (e.g., one or more Kinect cameras). The sensor device 103 may use a combination of sensors (e.g., the one or more sensors) to identify the user and provide information associated with the identified user to the electronic device 101. In an example, the sensor device 103 may be configured to detect motion data associated with the user and provide the motion data to the electronic device 101. In an example, the electronic device 101 may perform the calibrations for the virtual environment based on receiving the calibration data from the sensor device 103.

In an example, the sensor device 103 may be configured to detect objects or obstacles in front of the subject as the subject is performing the game via the headset 102. The sensor device 103 may be configured to alert the subject of any potential objects that may pose a safety risk to the subject while the subject is performing the movements while playing the game using the headset 102. For example, objects such as pets or people could pose a safety issue while the subject is performing one or more movements (e.g., moving the lower limbs) while using the headset 102. For example, the objects may be invisible to the sensors of the headset 102. Thus, the headset 102 may have difficulty detecting any potential hazardous objects that come within range of the subject's movements while the subject is using the headset 102. The sensor device 103 may be configured to use an object detection module for detecting objects (e.g., pets, people, etc.) within a radius of the headset 102. For example, when the object (e.g., pet, person, etc.) moves into a field of view of the sensor device 103, an alarm may be trigger to alert the subject of the object. For example, the headset 102 may output a pop-up panel within the image stream and a detection result. In an example, a predicted bounding box may be output identifying the object relative to the subject in the image stream output by the headset 102 to the subject.

The server 106 may include a group of one or more servers. In an example, all or some of the operations executed by the electronic device 101 may be executed in a different one or a plurality of electronic devices (e.g., the headset 102, the sensor device 103, or the server 106). In an example, if the electronic device 101 needs to perform a certain function or service either automatically or based on a request, the electronic device 101 may request at least some parts of functions related thereto alternatively or additionally to a different electronic device (e.g., the headset 102, the sensor device 103, or the server 106) instead of executing the function or the service autonomously. The different electronic devices (e.g., the headset 102, the sensor device 103, or the server 106) may execute the requested function or additional function, and may deliver a result thereof to the electronic device 101. The electronic device 101 may provide the requested function or service either directly or by additionally processing the received result. In an example, a cloud computing, distributed computing, or client-server computing technique may be used. For example, the electronic device 101 may provide the calibration data received from the sensor device 103 to the server 106, wherein the server 106 may perform the calibration operations and return the results to the electronic device 101

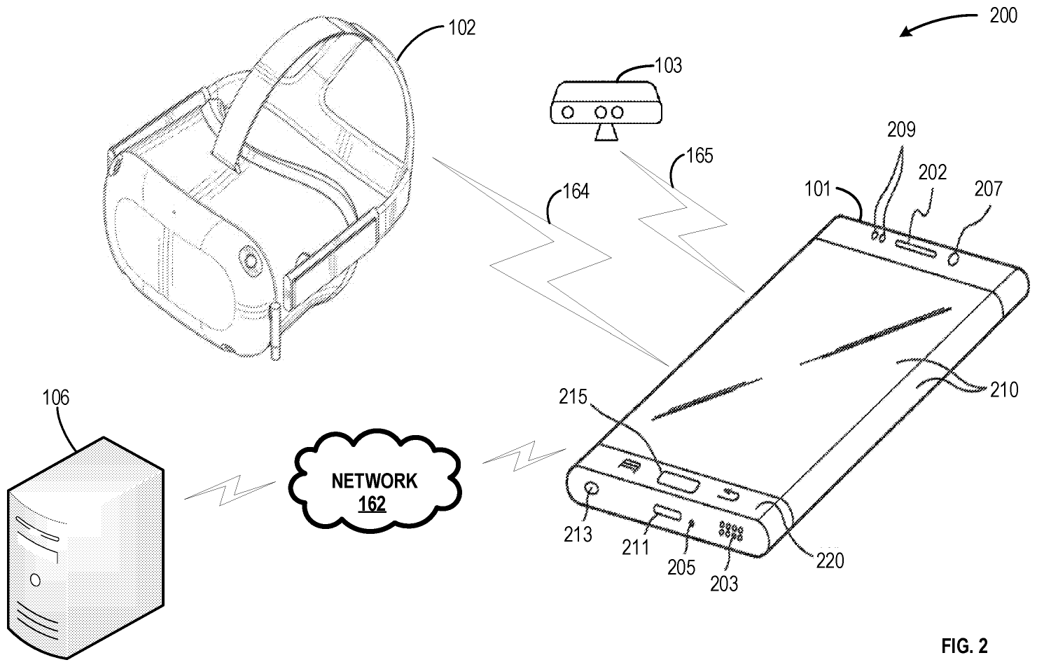

FIG. 2 shows an example system 200. The system 200 may comprise various components which may be in communication with some or other or all components. FIG. 2 shows an example system 200 wherein the electronic device 101 is in communication with the headset 102, the sensor device 103, and the server 106. The electronic device 101, and the headset 102 and the sensor device 103 may be communicatively coupled through a near field communication technology 164, 165 (e.g., Bluetooth Low Energy or WiFi). The electronic device 101 may be communicatively coupled to the server 106 through the network 162. The electronic device 101 may determine location information. For example the electronic device 101 may comprise a GPS sensor. The GPS sensor on the electronic device 101 may determine location information (e.g., GPS coordinates) and transmit the location information to the server 106.

The headset 102 may send data to the electronic device 101. The electronic device 101 may determine, via various sensors, image data, geographic data, orientation data, and the like. The electronic device 101 may further transmit said data to the server 106.

For example, the system 200 may comprise the electronic device 101, the headset 102, the sensor device 103, and the server 106 according to various embodiments of the present disclosure. The electronic device 101 and the headset 102 may be communicatively coupled to the server 106 via the network 162. In an example. the electronic device 101 may include a display 210, a housing (or a body) 220 to which the display 210 is coupled while the display 210 is seated therein, and an additional device formed on the housing 220 to perform the function of the electronic device 101. In an example, the additional device may include a first speaker 202, a second speaker 203, a microphone 205, sensors (e.g., a front camera module 207, a rear camera module, an illumination sensor 209, or the like). communication interfaces (e.g., a charging or data input/output port 211 and an audio input/output port 213), and a button 215. In an example, when the electronic device 101 and the headset 102 are connected through a wired communication scheme, the electronic device 101 and the headset 102 may be connected based on at least some ports (e.g., the data input/output port 211) of the communication interfaces.

In example. the display 210 may include a flat display or a bended display (or a curved display) which can be folded or bent through a paper-thin or flexible substrate without damage. The bended display may be coupled to a housing 220 to remain in a bent form. In an example, the mobile device 201 may be implemented as a display device, which can be quite freely folded and unfolded such as a flexible display, including the bended display. In an example, in a Liquid Crystal Display (LCD), a Light Emitting Diode (LED) display, an Organic LED (OLED) display, or an Active Matrix OLED (AMOLED) display, the display 210 may replace a glass substrate surrounding liquid crystal with a plastic film to assign flexibility to be folded and unfolded.

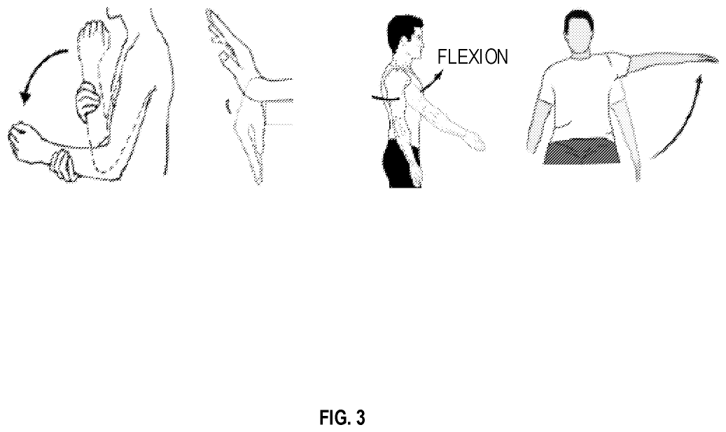

FIG. 3 shows example movements. The Virtual Remote Tele-Physical Examination (VIRTePEX) System may consist of two mixed reality scenes that function like exercise games in providing an interactive and engaging element to the remote assessment procedure. The primary focus may be on the upper body joints due to ease of tracking and lower possibility of noise and occlusions (unlike lower body joints such as ankles). As shown in FIG. 3, the VIRTePEX system may target three upper-body joints of the elbow, the wrist, and the shoulder for a total of four movements. The camera may be used for motion tracking and 3D skeleton inference. The VIRTePEX system may record the user motion for a specified joint in terms of a range of motion via the camera skeleton data and the time required to complete the motion via tracking hand gestures. The VIRTePEX system may estimate a force value for the motion using an inverse dynamics solver which may then be transmitted to virtual objects inside the game environment for providing an in-game feedback mechanism to a physician performing the remote assessment.

The VIRTePEX system may use a sensor device 103, such as a Kinect v2 depth camera. for tracking a subject's joint movements. Depth cameras provide 3D skeleton information of the subject in real-time which is useful for tracking and inferring parameters such as relative joint positions and angles. The force/torque on a joint may be estimated when a subject performs a specified movement. The range of motion and duration of the joint's movement from a specified start to end position may be used to estimate (up to a constant) the force required to perform the observed motion. For example, the procedure may involve capturing the motion of the human body using a depth camera to track the different joints and the associated angles over specified time intervals. The depth camera may not provide the values for joint angles directly. However, the values for the joint angles may be estimated using a simple dot product between the orientation vectors of the limb segments. The joint angles may then be used to compute kinematic quantities such as angular velocity and acceleration which are required for the force and torque estimations. which are provided through the inverse dynamics equations.

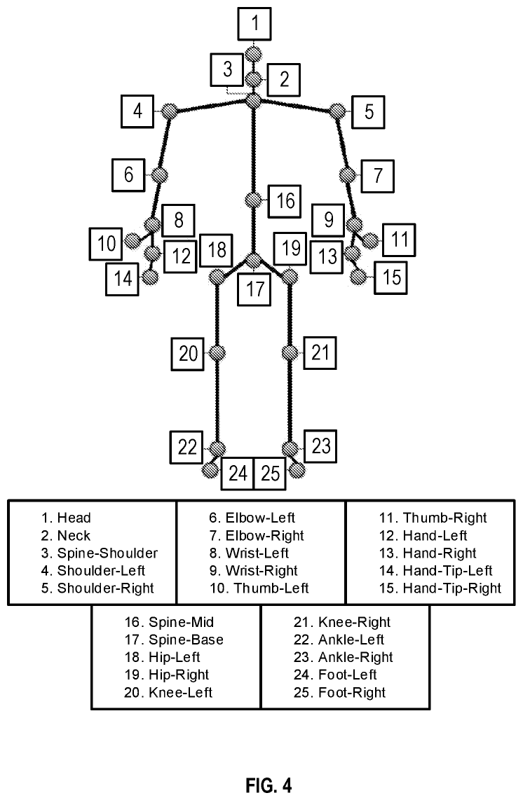

The joints may be modeled as a one link revolute joint since all of the considered movements are rotational in nature and there is only one degree of freedom in the overall movement consisting of the angular displacement at the joint. Although the raw force estimates may not be comparable in isolation with the ground truth values, they are consistent across movements corresponding to different strength levels. As shown in FIG. 3, three upper joints of the elbow, shoulder, and wrist are considered with flexion/extension movement for all of the joints and the abduction movement for the shoulder joint. From the skeleton provided by the depth camera, such as the one shown in FIG. 4, containing the 3D position data for the tracked body joints, the joint angles may be computed using a dot produced between the 3D position vectors.

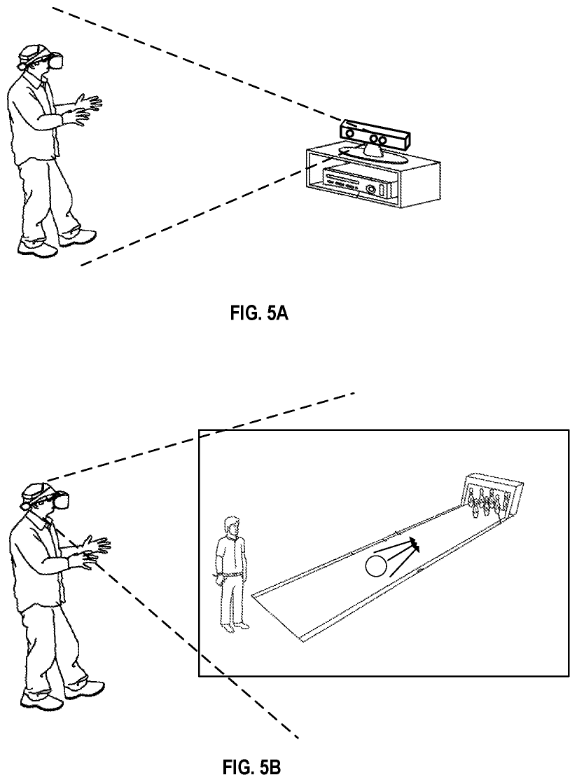

FIGS. 5A-5B show an example scenario. FIG. 5A shows an example scene with the user that may be captured by the sensor device 103. FIG. 5B shows an example virtual reality scene (e.g. environment) with a personalized humanoid avatar of the user, as the user is captured by the sensor device 103. As shown in FIG. 5B, the user may experience the virtual reality scene through the use of a headset device. FIG. 5B shows the user participating in an exercise game (e.g., bowling) as the VIRTePEX system estimates the force value for the user's motion while playing the game. For example, the exercise game may comprise a bowling game consisting of a virtual bowling alley scene with a ball and a bowling pin with variable mass, placed in-line along the alley. The variable pin weight may provide a means to evaluate the user in a standardized manner, based on a user's ability to knock pins from a predefined set of virtual weights. The bowling scene may allow for the system to target the upper body joints. The main objective for the user may be to perform the movement at his/her comfort level and observe whether the estimated force generated from the action can knock down the pin. While the user performs the activity, the joint angle and time from start to end may be tracked via the depth camera. At the end of the gaming session, the tracked joint data may be provided as input to the inverse dynamics solver to estimate the force which may be applied to the ball along the direction to the pin. The sensor device 103 may also generate motion data associated with the movements of the user which may be applied to the personalized humanoid avatar to simulate the user playing the game in the virtual environment.

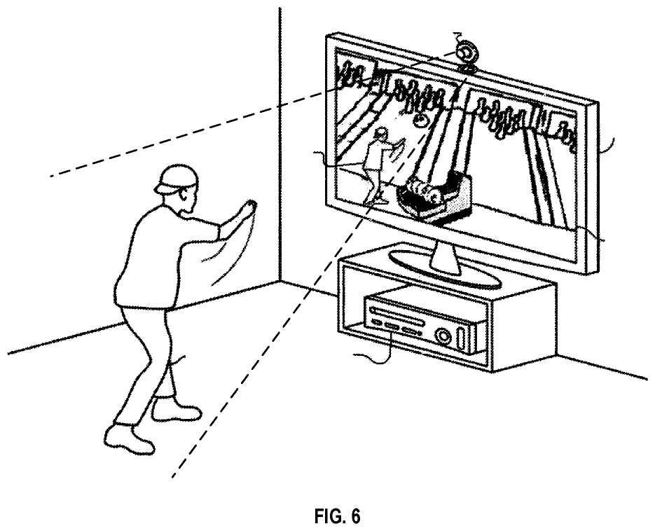

FIG. 6 shows an example scenario wherein the user uses a display device (e.g., television) to participate in the game. FIG. 6 shows the virtual reality scene (e.g., environment) being displayed from a display device (e.g., television). The user may participate in the exercise game (e.g., bowling) in the comfort of the user's living space. such as a living room or bedroom. The VIRTePEX system may utilize the sensor device 103 to estimate the force values associated with the user's joints, while the user interacts with the objects displayed during the game. Similarly to as shown in FIG. 5B, the user may participate in an exercise game comprising a bowling game consisting of a virtual bowling alley scene with a ball and a bowling pin with variable mass, placed in-line along the alley. The user may perform the movements at his/her comfort level and observe whether the estimated force generated from the action can knock down the pin. While the user performs the activity, the joint angle and time may each be tracked via the camera. At the end of the gaming session, the tracked joint data may be provided as input to the inverse dynamics solver to estimate the force which is applied to the ball along the direction to the pin. The sensor device 103 may capture movements of the user and generate motion data associated with the movements of the user. The motion data may then be applied to the personalized humanoid avatar to simulate the user playing the game in the virtual environment.

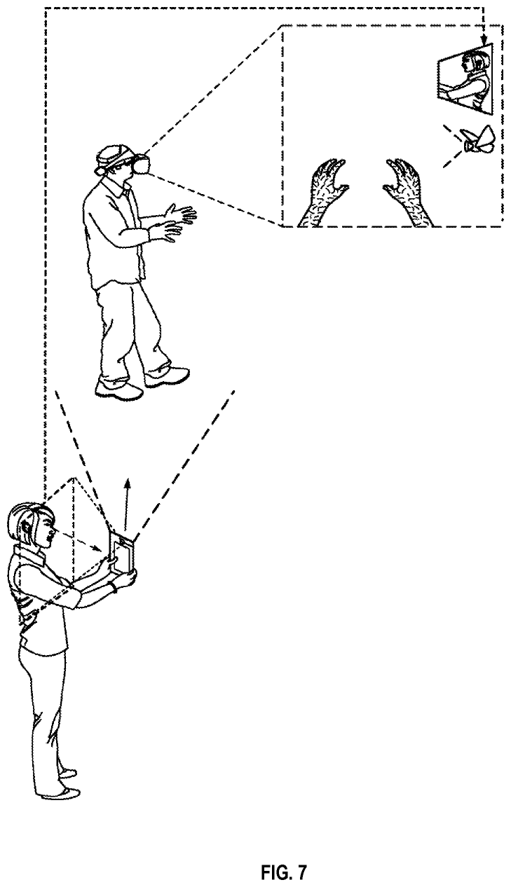

FIG. 7 shows an example scenario wherein the user may participate in the virtual exercise game while being remotely assessed by another person. such as the user's physician. The user's physician may remotely interact with the user in real-time as the user participates in the virtual exercise game in order to provide an assessment in real-time to the user. In an example, the user may interact with another user that is placed at an opposite end of the bowling alley. Both users may perform the required joint movement and the motion is imparted to each ball based on their respective force estimates, wherein the objective is to overcome the opposite user's ball in crossing the mid-point and observing the same in the resulting collision between the balls. In such an assessment, depending on the physical resistance that the user can overcome. the physician may then determine a judgement regarding the strength of the joint on a broad scale of three to five levels.

The user's physician may also interact with the user in an asynchronous manner, wherein the user's participation in the exercise game may be recorded and uploaded to a server. The user's physician may retrieve the user's records in order to assess the user's participation in the virtual exercise game. Furthermore, the records may provide a means for physicians to compare and benchmark joint strength data against other users. In an example, the users may be provided two options: recording his/her movement for future play; or play against another user's recording. During the recording mode, the user may perform the specified joint movement over multiple sessions, and during each session, the estimated force may be logged. With the non-recording mode. the user first specifies an identifier for the opposite user. The task may comprise attempting to overcome the opposing ball, which is a given force retrieved from the data logged during the opponent's recording. The asynchronous option enables users to play against his/her previous levels or a more personalized reference level by a physician. The asynchronous option may further enable the user to train against the previous levels or personalized reference level until he/she can achieve the muscular strength to exert the required force.

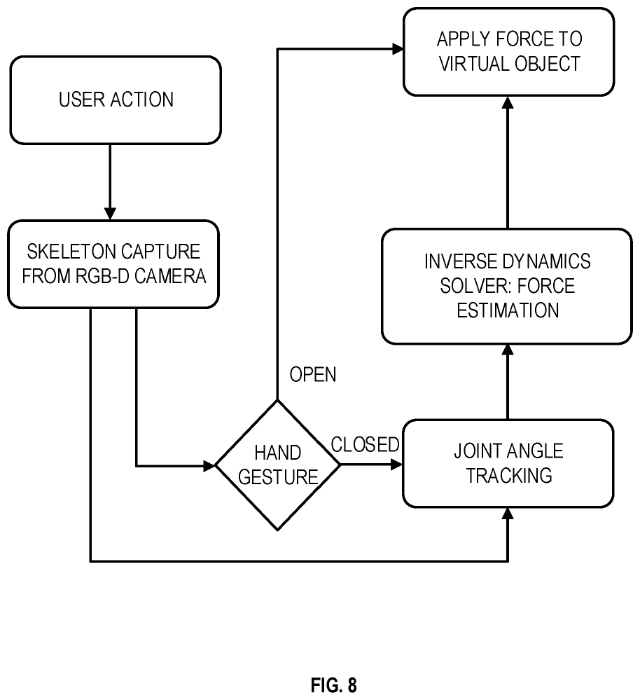

FIG. 8 shows an example process. Hand gesture controls may be utilized in order to control the system interface. For example, the hand gesture controls provided by the depth camera may be used to track the subject's intent to start and end an activity session. The user may signal the start and end of tracking the activity (e.g., elbow flexion) by closing and opening the hand, as shown in FIG. 8. A hand gesture-based control may correspond to the real-world actions of holding and releasing a ball which may comprise the mapping used in the virtual scene. A visual cue in the virtual scene may be provided in order to address any potential issues that may arise of a subject not knowing whether or not his/her gesture is registered. For example, a color of the virtual object (e.g., bowling ball) may be toggled when the start of an activity is detected.

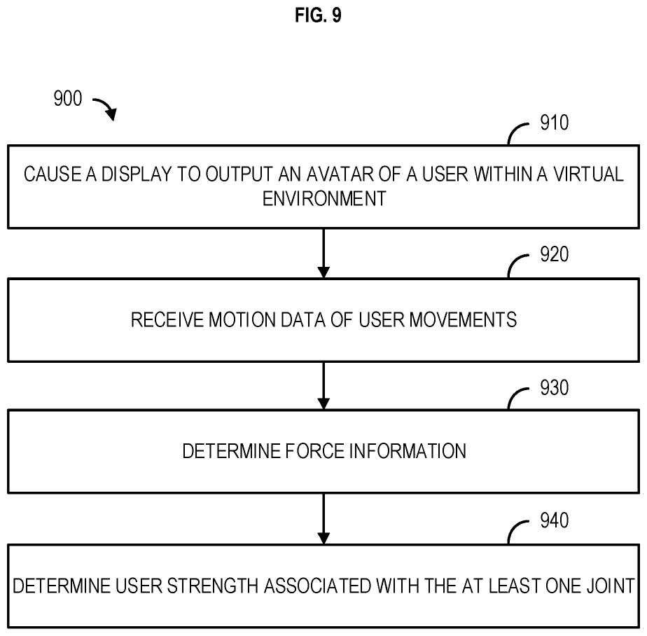

FIG. 9 shows a flow chart of an example method 900. The method 900 may be implemented in whole or in part, by one or more of, the electronic device 101, the headset 102, the sensor device 103, the server 106, or any other suitable device. At step 910, a display may output an avatar of the user within a virtual environment based on a calibration of a sensor. For example, the electronic device 101 may generate a personalized humanoid avatar, representative of the user, that may be output within the virtual environment displayed by the headset 102. For example, the process may include creating the skeletal joints and the associated texture information. The display may comprise at least one of a head mounted display, a television, a monitor, a laptop, or a tablet. In an example, the display may be further configured to output a game (e.g., virtual exercise game) to engage the user to interact with a virtual object within the virtual environment. For example, interacting with the virtual object within the virtual environment may further comprise causing the user to perform one or more movements.

As an example, the calibration of the sensor may be performed to estimate the floor plane for calibrating coordinates between the front of the sensor device 103 and the virtual environment if the skeletal joints of the user are fully detected. For example, a real-time camera-to-skeletal pose and a real-time floor calibration may be performed to estimate the floor plane of the environment detected in the initial data. For calibration purposes, it may be assumed that the user is standing or sitting in a vertical posture. This assumption may imply that the estimated joints corresponding to the spine are distributed about a vertical trend. Thus, this vertical trend may be used as floor normal for floor calibration. Furthermore, to ensure that the target user stays in a normal sitting or standing pose during calibration, the system may also track the shoulder height and knee angle. In an example, the user may adjust his/her position to interact with virtual objects in order to calibrate the sensors and/or headset/display 102.

As an example, orientation data may be determined. The orientation data may be associated with the headset 102. For example, the orientation data may comprise an indication of a 3D orientation of the headset 102. The orientation data may be determined based on the location of the center of the field of view of the headset 102. The orientation data may comprise an indication of a 3D orientation of the device (e.g., yaw, pitch, roll and the like). In an example, the orientation data may be determined by a sensor module included in the headset 102, such as a magnetic sensor, gyro sensor, accelerometer, or any combination thereof. In an example, the orientation may be determined based on data received by the sensor device 103. In an example, the orientation data may be associated with a display device instead of a headset 102.

At step 920, motion data of user movements may be received. For example, the electronic device 101 may receive motion data from the sensor device 103 as the user interacts with various virtual objects in the virtual environment, or during the virtual exercise game. The sensor device 103 may comprise a RGB-D camera for capturing images of the user for motion tracking and 3D skeleton inference. Based on the captured images, the sensor device 103 may generate motion data. For example, the captured images may be associated with the upper body joints due to ease of tracking and lower possibility of noise and occlusions (unlike lower body joints such as ankles). As an example, the motion data may be captured for three upper-body joints of the elbow, wrist, and shoulder for a total of four movements. The range of motion of specified joints may be tracked. For example, the motion data may include joint data associated with at least one joint of the user. For example, the joint data may be associated with the range of motion of the specified joints via the camera skeleton data and the time required to complete the motion. For example, values for the joint angles may be estimated using a simple dot product between the orientation vectors of the limb segments. The joint angles may then be used to compute kinematic quantities such as angular velocity and acceleration which are required for the force and torque estimations, which are provided through the inverse dynamics equations.

As an example, the electronic device 101 may apply the motion data to the personalized humanoid avatar. The electronic device 101 may cause the personalized humanoid avatar, output by the headset 102, to perform movements according to the movements captured by the sensor device 103 in real-time as the user moves. The motion data may be applied to the personalized humanoid avatar to simulate the user playing the game in the virtual environment.

At step 930, force information may be determined based on the joint data. As an example, an angle of the at least one joint may be tracked, based on the joint data, while the user interacts with the virtual object. The force information may be determined based on the tracked angle. The force information may comprise an estimation of a force acting on the at least one joint while the user interacts with the virtual object during the game. For example, the electronic device 101 may estimate a force value for the motion using an inverse dynamics solver which is then transmitted to virtual objects inside the game environment for providing an in-game feedback mechanism. For example, the feedback may be provided to a physician performing remote assessment of the user, The electronic device 101 may use the motion data, including the tracked joint data, as input to an inverse dynamics solver to estimate the force and torque applied to a virtual object as the user interacts with the virtual object during an exercise gaming session. In an example, the estimated force may be applied to the virtual object.

As an example, second motion data associated with user movements may be received from a second sensor. The second motion data may comprise second joint data associated with at least one joint of a second user. Second force information may be determined based on the second joint data. The second force information may comprise an estimation of a force acting on the at least one joint of the second user while the second user interacts with a second virtual object within the virtual environment. In an example, the virtual object may be caused to overcome the second virtual object based on the force information and the second force information. In an example, the second virtual object may be caused to overcome the virtual object based on the force information and the second force information.

As an example, the motion data and the data associated with the user interacting with the virtual object within the virtual environment may be output to a communication network for remote access. For example, the force information may further comprise a force perception association with the output of the motion data and the data associated with the user interacting with the virtual object within the virtual environment. As an example, data comprising the force information may be sent to a server. The server may associate the data with the user and may store the data associated with the user in a database.

At step 940, user strength associated with the at least one joint may be determined based on the force information. For example, the electronic device 101 may determine the user strength associated with the at least one joint based on the force information. As an example, data associated with the user strength associated with the at least one joint may be sent to a server as joint strength data. The joint strength data may be associated with the user in order to improve the quality of the user's experience during the entire process and track the joint strength estimations for the user. For example, a user's physician may use the joint strength data of the user to make further assessments regarding the user's joints.

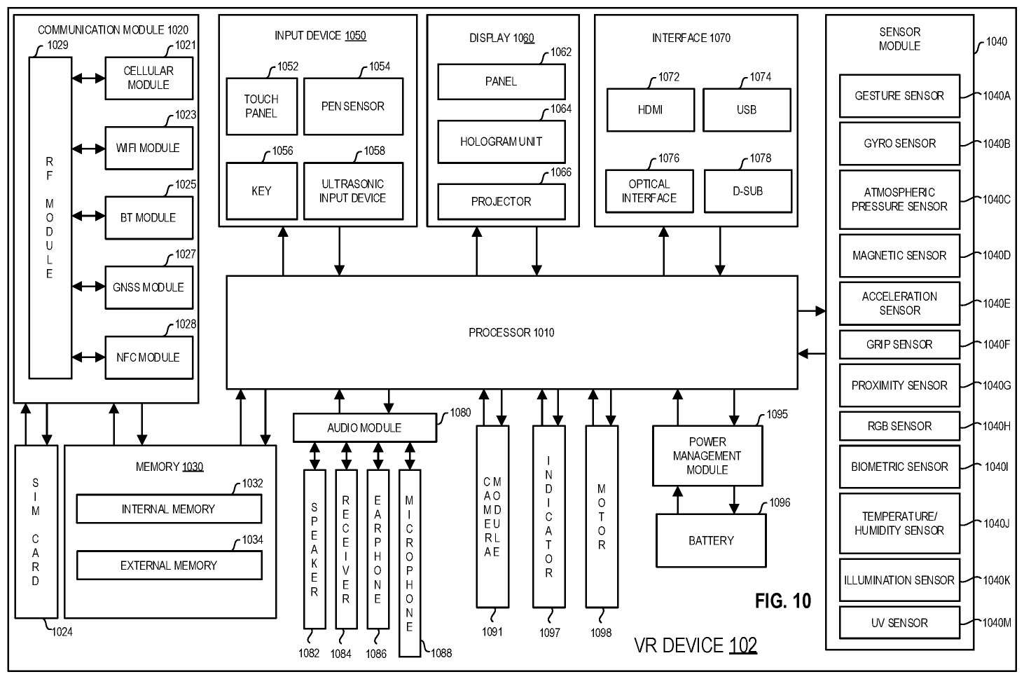

FIG. 10 shows a block diagram of a headset device 102 according to various exemplary embodiments. The headset device 102 may include one or more processors (e.g., Application Processors (APs)) 1010, a communication module 1020, a subscriber identity module 1024, a memory 1030, a sensor module 1040, an input unit 1050, a display 1060, an interface 1070, an audio module 1080, a camera module 1091, a power management module 1095, a battery 1096, an indicator 1097, and a motor 1098. Camera module 1091 may comprise an aperture configured for a change in focus.

The processor 1010 may control a plurality of hardware or software constitutional elements connected to the processor 1010 by driving, for example, an operating system or an application program, and may process a variety of data including multimedia data and may perform an arithmetic operation (for example, distance calculations), For instance, the processor 1010 may be configured to generate a personalized humanoid avatar of the subject and place the personalized humanoid avatar within a virtual reality scene, for example the mixed reality scene shown in FIGS. 5B and 6. The processor 1010 may be implemented. for example, with a System on Chip (SoC). According to one exemplary embodiment, the processor 1010 may further include a Graphic Processing Unit (GPU) and/or an Image Signal Processor (ISP). The processor 1010 may include at least one part (e.g., a cellular module 1021) of the aforementioned constitutional elements of FIG. 10. The processor 1010 may process an instruction or data, for example the mixed reality program 147, which may be received from at least one of different constitutional elements (e.g., a non-volatile memory), by loading it to a volatile memory and may store a variety of data in the non-volatile memory. The processor may receive inputs such as sensor readings and execute the augmented reality program 147 accordingly by, for example, adjusting the position of the virtual object within the augmented reality scene. For example, the processor 1010 might adjust the position and the orientation of the personalized humanoid avatar within the virtual environment.

The communication module 1020 may include, for example, the cellular module 521, a Wi-Fi module 1023, a BlueTooth (BT) module 1025, a GNSS module 1027 (e.g., a GPS module, a Glonass module, a Beidou module, or a Galileo module), a Near Field Communication (NFC) module 1028, and a Radio Frequency (RF) module 1029. The communication module may receive data from the electronic device 101, the sensor device 103, and/or the server 106. The communication module may transmit data to the electronic device 101 and/or the server 106. In an exemplary configuration, the headset device 102 may transmit data determined by the sensor module 1040 to the electronic device 101 and/or the server 106. For example, the headset device 102 may transmit, to the electronic device 101, via the BT module 1025, data gathered by the sensor module 1040.

The cellular module 1021 may provide a voice call, a video call, a text service, an internet service, or the like, for example, through a communication network. According to one exemplary embodiment, the cellular module 1021 may identify and authenticate the headset device 102 in the network 162 by using the subscriber identity module (e.g., a Subscriber Identity Module (SIM) card) 1024. According to one exemplary embodiment, the cellular module 1021 may perform at least some functions that can be provided by the processor 1010. According to one exemplary embodiment, the cellular module 1021 may include a Communication Processor (CP).

Each of the WiFi module 1023, the BT module 1025, the GNSS module 1027, or the NFC module 1028 may include, for example, a processor for processing data transmitted/received via a corresponding module. According to a certain exemplary embodiment, at least some (e.g., two or more) of the cellular module 1021, the WiFi module 1023, the BT module 1025, the GPS module 1027, and the NFC module 1028 may be included in one Integrated Chip (IC) or IC package. The GPS module 1027 may communicate via network 162 with the electronic device 101, the server 106, or some other location data service to determine location information, for example GPS coordinates. The RF module 1029 may transmit/receive, for example, a communication signal (e.g., a Radio Frequency (RF) signal). The headset device 102 may transmit and receive data from the mobile device via the RF module 1029. Likewise, the headset device 102 may transmit and receive data from the server 106 via the RF module 1029. The RF module may transmit a request for location information to the server 106. The RF module 1029 may include, for example, a transceiver, a Power Amp Module (PAM), a frequency filter, a Low Noise Amplifier (LNA), an antenna, or the like. According to another exemplary embodiment, at least one of the cellular module 1021, the WiFi module 1023, the BT module 1025, the GPS module 1027, and the NFC module 1028 may transmit/receive an RF signal via a separate RF module.

The subscriber identity module 1024 may include, for example, a card including the subscriber identity module and/or an embedded SIM, and may include unique identification information (e.g., an Integrated Circuit Card IDentifier (ICCID)) or subscriber information (e.g., an International Mobile Subscriber Identity (IMSI)).

The memory 1030 (e.g., the memory 130) may include, for example, an internal memory 1032 or an external memory 1034. The internal memory 1032 may include, for example, at least one of a volatile memory (e.g., a Dynamic RAM (DRAM), a Static RAM (SRAM), a Synchronous Dynamic RAM (SDRAM), etc.) and a non-volatile memory (e.g., a One Time Programmable ROM (OTPROM), a Programmable ROM (PROM), an Erasable and Programmable ROM (EPROM), an Electrically Erasable and Programmable ROM (EEPROM), a mask ROM, a flash ROM, a flash memory (e.g., a NAND flash memory, a NOR flash memory, etc.). a hard drive, or a Solid State Drive (SSD)).

The external memory 1034 may further include a flash drive, for example, Compact Flash (CF), Secure Digital (SD), Micro Secure Digital (Micro-SD), Mini Secure digital (Mini-SD), extreme Digital (xD), memory stick, or the like. The external memory 1034 may be operatively and/or physically connected to the headset device 102 via various interfaces.

The sensor module 1040 may measure, for example, a physical quantity or detect an operational status of the headset device 102, and may convert the measured or detected information into an electric signal. The sensor module 1040 may include, for example, at least one of a gesture sensor 1040A, a gyro sensor 1040B, a pressure sensor 1040C, a magnetic sensor 1040D, an acceleration sensor 1040E, a grip sensor 1040F, a proximity sensor 1040G, a color sensor 1040H (e.g., a Red. Green, Blue (RGB) sensor), a bio sensor 1040I, a temperature/humidity sensor 1040J, an illumination sensor 1040K, an Ultra Violet (UV) sensor 1040M, an ultrasonic sensor 1040N, and an optical sensor 1040P. Proximity sensor 1040G may comprise LIDAR, radar, sonar, time-of-flight, infrared or other proximity sensing technologies. The gesture sensor 1040A may determine a gesture associated with the headset device 102. For example, as the headset device 102 moves within the mixed reality scene, the headset device 102 may move in a particular way so as to execute, for example, a game action. The gyro sensor 1040B may be configured to determine a manipulation of the headset device 102 in space, for example when the headset device 102 is located on a user's head, the gyro sensor 1040B may determine the user has rotated the user's head a certain number of degrees. Accordingly. the gyro sensor 1040B may communicate a degree of rotation to the processor 1010 so as to adjust the mixed reality scene by the certain number of degrees and accordingly maintaining the position of, for example, the personalized avatar, or a virtual object, as rendered within the mixed reality scene. The proximity sensor 1040G may be configured to use sonar, radar, LIDAR, or any other suitable means to determine a proximity between the headset device and one or more physical objects. The ultrasonic sensor 1040N may also be likewise configured to employ sonar, radar, LIDAR, time of flight, and the like to determine a distance. The ultrasonic sensor may emit and receive acoustic signals and convert the acoustic signals into electrical signal data. The electrical signal data may be communicated to the processor 1010 and used to determine any of the image data, spatial data, or the like. According to one exemplary embodiment, the optical sensor 1040P may detect ambient light and/or light reflected by an external object (e.g., a user's finger. etc.). and which is converted into a specific wavelength band by means of a light converting member. Additionally or alternatively, the sensor module 1040 may include, for example, an E-nose sensor, an ElectroMyoGraphy (EMG) sensor, an ElectroEncephaloGram (EEG) sensor, an ElectroCardioGram (ECG) sensor, an Infrared (IR) sensor, an iris sensor, and/or a fingerprint sensor. The sensor module 1040 may further include a control circuit for controlling at least one or more sensors included therein. In a certain exemplary embodiment, the headset device 102 may further include a processor configured to control the sensor module 1004 either separately or as one part of the processor 1010. and may control the sensor module 1040 while the processor 1010 is in a sleep state.

The input device 1050 may include, for example, a touch panel 1052, a (digital) pen sensor 1054, a key 1056, or an ultrasonic input device 1058. The touch panel 1052 may recognize a touch input, for example, by using at least one of an electrostatic type, a pressure-sensitive type, and an ultrasonic type. In addition, the touch panel 1052 may further include a control circuit. The touch panel 1052 may further include a tactile layer and thus may provide the user with a tactile reaction.

The (digital) pen sensor 1054 may be, for example, one part of a touch panel, or may include an additional sheet for recognition. The key 1056 may be, for example, a physical button, an optical key, a keypad, or a touch key. The ultrasonic input device 1058 may detect an ultrasonic wave generated from an input means through a microphone (e.g., a microphone 1088) to confirm data corresponding to the detected ultrasonic wave.

The display 1060 (e.g., the display 1060) may include a panel 1062, a hologram unit 1064, or a projector 1066. The panel 1062 may include a structure the same as or similar to the display 210 of FIG. 2. The panel 1062 may be implemented, for example, in a flexible, transparent, or wearable manner. The panel 1062 may be constructed as one module with the touch panel 1052. According to one exemplary embodiment, the panel 1062 may include a pressure sensor (or a force sensor) capable of measuring strength of pressure for a user's touch. The pressure sensor may be implemented in an integral form with respect to the touch panel 1052, or may be implemented as one or more sensors separated from the touch panel 1052.

The hologram unit 1064 may use an interference of light and show a stereoscopic image in the air. The projector 1066 may display an image by projecting a light beam onto a screen. The screen may be located, for example, inside or outside the headset device 102. According to one exemplary embodiment, the display 1060 may further include a control circuit for controlling the panel 1062, the hologram unit 1064, or the projector 1066.

The display 1060 may display a real-world scene and/or the mixed reality scene. The display 1060 may receive image data captured by camera module 1091 from the processor 1010. The display 1060 may display the image data. The display 1060 may display the one or more physical objects. The display 1060 may display one or more virtual objects such as a virtual ball, virtual animal, virtual furniture, etc. The user may interact with the one or more virtual objects, wherein the user may adjust his/her position in the virtual environment, if necessary, and reach for the virtual objects.

The interface 1070 may include, for example, a High-Definition Multimedia Interface (HDMI) 1072, a Universal Serial Bus (USB) 1074, an optical communication interface 1076, or a D-subminiature (D-sub) 1078. The interface 1070 may be included, for example, in the communication interface 170 of FIG. 1. Additionally or alternatively, the interface 1070 may include, for example, a Mobile High-definition Link (MHL) interface, a Secure Digital (SD)/Multi-Media Card (MMC) interface, or an Infrared Data Association (IrDA) standard interface.

The audio module 1080 may bilaterally convert, for example, a sound and electric signal. At least some constitutional elements of the audio module 1080 may be included in, for example, the input/output interface 150 of FIG. 1. The audio module 1080 may convert sound information which is input or output, for example, through a speaker 1082, a receiver 1084, an earphone 1086, the microphone 1088, or the like.

The camera module 1091 is, for example, a device for image and video capturing, and according to one exemplary embodiment. may include one or more image sensors (e.g., a front sensor or a rear sensor), a lens, an Image Signal Processor (ISP), or a flash (e.g., LED or xenon lamp), The camera module 1091 may comprise a forward facing camera for capturing a scene. The camera module 1091 may also comprise a rear-facing camera for capturing eye-movements or changes in gaze.

The power management module 1095 may manage, for example, power of the headset device 102. According to one exemplary embodiment, the power management module 1095 may include a Power Management Integrated Circuit (PMIC), a charger Integrated Circuit (IC), or a battery fuel gauge. The PMIC may have a wired and/or wireless charging type. The wireless charging type may include, for example, a magnetic resonance type, a magnetic induction type, an electromagnetic type, or the like, and may further include an additional circuit for wireless charging, for example, a coil loop, a resonant circuit, a rectifier, or the like. The battery gauge may measure, for example, residual quantity of the battery 1096 and voltage, current, and temperature during charging. The battery 1096 may include, for example, a rechargeable battery and/or a solar battery.

The indicator 1097 may display a specific state, for example, a booting state, a message state, a charging state, or the like, of the headset device 102 or one part thereof (e.g., the processor 1010). The motor 1098 may convert an electric signal into a mechanical vibration, and may generate a vibration or haptic effect. Although not shown, the headset device 102 may include a processing device (e.g., a GPU) for supporting a mobile TV. The processing device for supporting the mobile TV may process media data conforming to a protocol of, for example, Digital Multimedia Broadcasting (DMB), Digital Video Broadcasting (DVB), MediaFlo™, or the like.

For purposes of illustration, application programs and other executable program components are illustrated herein as discrete blocks, although it is recognized that such programs and components can reside at various times in different storage components. An implementation of the described methods can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly. where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method comprising:
causing a display, based on a calibration of a sensor, to output an avatar of a user within a virtual environment;
receiving, from the sensor, motion data of user movements, wherein the motion data comprises joint data associated with at least one joint of the user;
determining, based on the joint data, force information; and
determining, based on the force information, user strength associated with the at least one joint.

2. The method of claim 1, wherein the display comprises a head mounted display (HMD), a television, a monitor, a laptop, or a tablet.

3. (canceled)

4. The method of claim 1, wherein the sensor comprises a RGB-D camera.

5. (canceled)

6. The method of claim 1, further comprising causing the display to output a game to engage the user to interact with a virtual object within the virtual environment.

7. (canceled)

8. The method of claim 6, wherein determining, based on the joint data, the force information comprises:
tracking, based on the joint data, an angle of the at least one joint while the user interacts with the virtual object; and
determining, based on the tracked angle, the force information.

9. The method of claim 6, wherein the force information comprises an estimation of a force acting on the at least one joint while the user interacts with the virtual object during the game.

10. The method of claim 9, further comprising:
receiving, from a second sensor, second motion data of user movements, wherein the second motion data comprises second joint data associated with at least one joint of a second user;
determining, based on the second joint data, second force information, wherein the second force information comprises an estimation of a force acting on the at least one joint of the second user while the second user interacts with a second virtual object within the virtual environment; and
causing, based on the force information and the second force information, the virtual object to overcome the second virtual object.

11. (canceled)

12. The method of claim 6, further comprising outputting the motion data and data associated with the user interacting with the virtual object within the virtual environment to a communication network for remote access.

13. The method of claim 12, wherein the force information comprises a force perception association with the output of the motion data and the data associated with the user interacting with the virtual object within the virtual environment.

14. (canceled)

15. The method of claim 1, wherein the calibration of the sensor comprises performing a real-time camera-to-skeletal pose calibration.

16. An apparatus comprising:
one or more processors; and
a memory storing processor-executable instructions that, when executed by the one or more processors, cause the apparatus to:
cause a display, based on a calibration of a sensor, to output an avatar of a user within a virtual environment;
receive, from the sensor, motion data of user movements, wherein the motion data comprises joint data associated with at least one joint of the user;
determine, based on the joint data, force information; and
determine, based on the force information, user strength associated with the at least one joint.

17. The apparatus of claim 16, wherein the display comprises a head mounted display (HMD), a television, a monitor, a laptop, or a tablet.

18. (canceled)

19. The apparatus of claim 16, wherein the sensor comprises a RGB-D camera.

20. (canceled)

21. The apparatus of claim 16, wherein the processor executable instructions, when executed by the one or more processors, further cause the apparatus to cause the display device to output a game to engage the user to interact with a virtual object within the virtual environment.

22. (canceled)

23. The apparatus of claim 21, wherein the processor-executable instructions that, when executed by the one or more processors, cause the apparatus to determine, based on the joint data, the force information further cause the apparatus to:
track, based on the joint data, an angle of the at least one joint while the user interacts with the virtual object; and
determine, based on the tracked angle, the force information.

24. The apparatus of claim 21, wherein the force information comprises an estimation of a force acting on the at least one joint while the user engages the virtual object during the game.

25. The apparatus of claim 24, wherein the processor-executable instructions, when executed by the one or more processors, further causes the apparatus to:
receive, from a second sensor, second motion data of user movements, wherein the second motion data comprises second joint data associated with at least one joint of a second user;
determine, based on the second joint data, second force information, wherein the second force information comprises an estimation of a force acting on the at least one joint of the second user while the second user interacts with a second virtual object within the virtual environment; and
cause, based on the force information and the second force information, the virtual object to overcome the second virtual object.

26. (canceled)

27. The apparatus of claim 21, wherein processor-executable instructions, when executed by the one or more processors, further causes the apparatus to output the motion data and data associated with the user interacting with the virtual object within the virtual environment to a communication network for remote access.

28. The apparatus of claim 27, wherein the force information comprises a force perception association with the output of the motion data and the data associated with the user interacting with the virtual object within the virtual environment.

29. (canceled)

30. The apparatus of claim 16, wherein the calibration of the sensor comprises performing a real-time camera-to-skeletal pose calibration.
